(19) United States
(12) Patent Application Publication
(10) Pub. No.: US 2011/0165613 A1
(43) Pub. Date: Jul. 7, 2011

(54) STABLE CLONE CELL EXPRESSING A PRION

(75) Inventor: Bruno You, L'Hay les Rose (FR)

(73) Assignees: LFB-Biotechnologies, Les Ulis (FR); Institut National de la Recherche Agronomique, Paris Cedex 07 (FR)

(21) Appl. No.: 13/062,475

(22) PCT Filed: Sep. 4, 2009
(86) PCT No.: PCT/FR09/51673

§ 371 Date: Mar. 4, 2011

(30) Foreign Application Priority Data
Sep. 5, 2008 (FR) 0855985

(51) Int. Cl. C12Q 1/18; C12Q 1/02; C12N 1/00

(52) U.S. Cl. 435/32; 435/29; 435/243

(57) ABSTRACT

The invention relates to a cell clone derived from the MovS6 line, said cell clone expressing a prion protein PrP and being capable of tolerating the replication or propagation of the pathological form PrPsc of said PrP, characterized in that its titre with respect to marker for infection with a non-conventional transmissible agent (NCTA) is stable at least up to the 6^{th} passage.

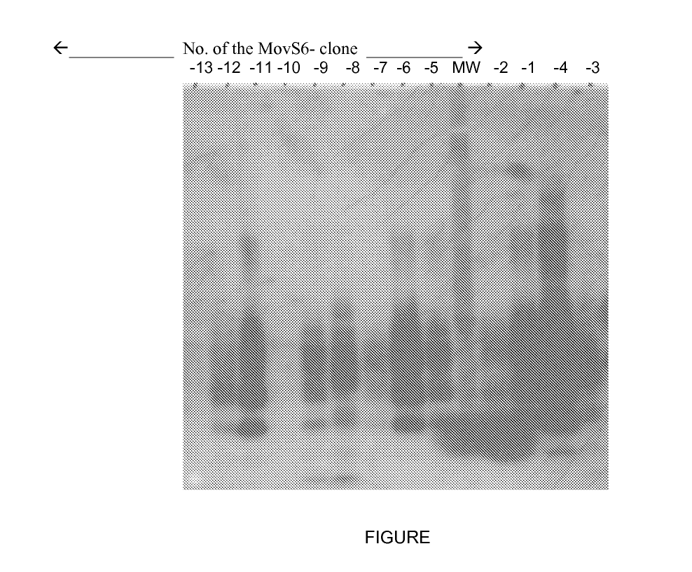

The present invention relates to a cell clone derived from the MovS6 line expressing a prion protein PrP and capable of withstanding the replication or propagation of the pathological form PrPsc of said PrP, and also the use thereof, in particular in an in vitro method for evaluating and/or checking the effectiveness of a process for obtaining or treating a biological product, or in an in vitro method for evaluating and/or checking a decontamination procedure, or in a method for evaluating or screening compounds which have an activity that modulates infectiousness related to “non-conventional transmissible agents”, NCTAs.

### PRIOR ART

Transmissible spongiform encephalopathies (TSEs) group together a collection of genetic or acquired diseases characterized by degeneration of the central nervous system (CNS). The most common form in humans is Creutzfeldt-Jakob disease (CJD), but TSEs also exist in many mammals (in particular scrapie in sheep and bovine spongiform encephalopathy). The aetiological agent of these diseases is classified in the category of “non-conventional transmissible agents” (NCTAs). The signature of the disease is the presence of an extracellular protein, called prion protein (PrP), which is converted during the disease into an insoluble form that is resistant to proteases such as proteinase K, and which accumulates in the central nervous system. This pathological abnormal form of PrP, called PrPsc, is copurified with infectiousness and its accumulation precedes the appearance of histological lesions. It results from a modification of the conformation of the PrP prion protein. No modification of the expression of the gene encoding PrP has been demonstrated, nor has any impairment of its translation (Prusiner, Biochemistry 1992; 31: 12277-88).

The data currently available do not make it possible to demonstrate that the transmissible agent responsible for TSEs is in an infecting form in blood derivatives (Brown et al., 2001, Semin Hematol.; 38 (4 Suppl 9): 2-6).

However, it cannot be concluded that it is absent, this uncertainty resulting, firstly, from the probable very low concentration in the blood and, secondly, from the very long clinically silent period of incubation characteristic of these diseases, which precedes the appearance of clinical signs.

In addition, the exceptional resistance of NCTAs prevents recourse to methods of inactivation conventionally used, such as Tween-TNBP solvent/detergent treatment, which have been proven to be effective in reducing the viral load of blood derivatives, such as cryoprecipitated plasma proteins (factor VIII, von Willebrand factor, etc.). Given that the obtaining or the treatment of biological products, such as plasma clotting products, should incorporate viral elimination/inactivation steps in view of a therapeutic use, the pharmaceutical industry for blood-derived medicaments today seeks to evaluate the theoretical risk of transmission of variant CJD by blood-derived products.

Currently, the method for the titration of NCTA-related infectiousness conventionally employed uses an in vivo titration method in the golden hamster, by intracerebral injection of various dilutions of an NCTA-loaded test product. Depending on the number of animals affected in the various groups corresponding to the dilutions carried out, it is possible to calculate an infectious titre and to establish the reduction factor of a given process on the basis of an untreated reference. However, this method has the drawback of being long (approximately one year), expensive and relatively incompatible with industrial-scale development, which requires a quick result regarding prion elimination effectiveness.

In addition, it is often necessary to introduce a step for concentrating the infectious agent so as to increase the sensitivity of the titration methods. All the procedures for concentrating infectious agents responsible for TSEs nowadays involve purification of PrPsc.

Other methods have been proposed for the in vitro titration of TSE infectious agents.

Techniques for detecting PrPsc by Western blotting (MacGregor, Transfusion J. Medecine 2001; 11, 3-14) or by ELISA generally require prior digestion of the sample to be analysed, with proteinase K, or denaturation with chaotropic agents in order to distinguish the pathological protein (PrPsc) from the normal protein (PrP).

Another titration method has recently been developed, based on the use of PrPsc-specific antibodies which do not recognize PrP (Korth et al, Nature 1997 Nov. 6, 390 (6655): 74-7).

U.S. Pat. No. 6,150,583 describes, for its part, the production of transgenic animals expressing a PrP labelled with a heterologous epitope which is or is not exposed at the surface of the prion protein, depending on the conformation of the latter.

The process called “PMCA” (Protein misfolding cyclic amplification), described in the document Saborio et al. (Nature; 2001, 411, 810-3), envisages bringing pathological forms originating from a tissue or a fluid derived from a contaminated animal into contact with a nonpathological form of the NCTA protein, in order to convert the latter into a pathological form. The PMCA method, although still in development, proves to be at least as sensitive as the bioassay. Nevertheless, it provides no evidence regarding the infectiousness of the PrPsc detected and proves difficult to implement with plasma matrices. In addition, uncertain reproducibility and false-positive results have been reported.

Document WO 2005/022148 describes a method for the in vitro titration, called “TCIA” (“tissue culture infectivity assay”), of an NCTA in a biological product, by means of bringing stable transgenic cells which tolerate replication of said NCTA into contact with the biological product, and then culturing these cells for one or more passages in order to amplify the amount of NCTA present in the biological product by replication of the NCTA. More specifically, the TCIA consists in bringing successive dilutions of an infectious homogenate (for example, scrapie strain 127-S) into contact with cells in culture on a multiwell plate, in a proportion of 5 wells per dilution. The number of positive wells is then evaluated by detection of PrPsc (marker indissociable from infectiousness) after 8 to 10 passages, and the titre is determined by the Spearman-Karber method.

The reproducibility of the titres obtained from the same infectious sample (brain homogenate) shows the feasibility of such an approach. However, the cell line used in this in vitro titration method, namely the MovS6 line, consists of a heterogeneous population of cells. In the end, this heterogeneity is liable to impair the stability of the line and, consequently, the reproducibility of the TCIA method.

There remains, therefore, a great need to have one or more stable clones derived from the MovS6 line in order to improve the stability of the TCIA method over time.

### SUMMARY OF THE INVENTION

The invention now provides a novel cell clone derived from the MovS6 line expressing a prion protein PrP and capable of withstanding the replication or propagation of the pathological form PrPsc of said PrP, characterized in that its PrPsc production is stable at least up to the 6^{th} passage, preferably over a period at least between the 6^{th} and the 100^{th} passage.

The inventors have in fact demonstrated that it is possible to have a clone which responds strongly to infection and which shows PrPsc production stability, i.e. without significant variation in the level of PrPsc production.

Preferably, the cell lysate derived from an infected culture of this clone has, starting from 6 weeks after the infection, an infection marker titre greater than or equal to 3 log₁₀ TCID50 per million cells, preferably greater than or equal to 4 log₁₀ TCID50 per million cells, preferably greater than or equal to 6 log₁₀ TCID50 per million cells, preferably greater than 7 log₁₀ TCID50 per million cells.

In one particular embodiment of the invention, the cell lysate of an infected culture has, starting from 6 weeks, an infection marker titre greater than 3 log₁₀ TCID50/million cells, preferably between 4.5 log₁₀ TCID50 and 6 log₁₀ TCID50 per million cells.

In one particular embodiment of the invention, the cell lysate of an infected culture has, starting from 6 weeks, an infection marker titre greater than 3 log₁₀ TCID50 per million cells, preferably between 4.5 log₁₀ TCID50 and 5.5 log₁₀ TCID50 per million cells.

In another embodiment, the cell lysate derived from an infected culture of this clone has, from 6 weeks after infection, an infection marker titre greater than or equal to 2 log₁₀ Western blotting units/million cells (uWB/million cells), preferably greater than or equal to 3 log₁₀ uWB/million cells.

Particularly preferably, the cell clone of the invention is denoted MovS6-4 in the experimental results disclosed below, and was deposited on 22 Feb. 2008, under number CNCM 1-3922, with the Collection Nationale de Cultures des Microorganismes [National Collection of Microorganism Cultures] (CNCM, Institut Pasteur, 25 rue du Docteur Roux, F-75724 Paris Cedex 15, France).

The invention is also directed towards an in vitro method for detecting and/or titrating the infectiousness of a non-conventional transmissible agent (NCTA), the marker of which is a protein of pathological conformation, in a sample, comprising the steps consisting in:
i) bringing said sample into contact with a cell clone as described above,
ii) culturing said cell clones in order to amplify the amount of NCTA present in said sample by replication of said NCTA,
iii) determining the presence and/or the amount of the NCTA in the sample, the culturing step ii) being carried out for one or more passages.

Advantageously, step (iii) comprises the following steps:
a) bringing the NCTA thus amplified at the end of step ii) into contact with a source of substrate for the PrP and/or the NCTA,
b) incubating the reaction medium allowing the conversion of the non-pathological conformer of the PrP into the pathological conformer and/or the amplification of the NCTA,
c) disaggregating the aggregates possibly formed during step i) or ii),
d) determining the presence and/or the amount of the NCTA in the sample, steps (a) to (c) constituting a cycle of operations which is repeated at least twice before step (d).

Another subject of the invention is an in vitro method for evaluating and/or checking a process for obtaining or treating a biological product or a material that may be contaminated with an NCTA, in which method a titration method as defined above is applied to said biological product or material, (A) upstream and (B) downstream of said process, and the two titre values (A) and (B) obtained are compared.

The invention is also directed towards an in vitro method for evaluating and/or checking a procedure for decontamination of a biological product or of a material, in which method a titration method as defined above is applied to said biological product or material, (A) upstream and (B) downstream of said procedure, and the two titre values (A) and (B) obtained are compared.

Yet another subject of the invention is an in vitro method for evaluating a compound capable of inhibiting the infectiousness of an infectious biological product, in which method a titration method as defined above is applied to said infectious biological product, (A) in the presence and (B) in the absence of said compound to be evaluated, and the two titre values (A) and (B) obtained are compared.

Yet another subject of the invention is an in vitro method for the diagnosis of a transmissible spongiform encephalopathy in a human subject or a non-human animal subject, which comprises detecting the presence, in a biological sample from said subject, of a non-conventional transmissible agent (NCTA) which is a protein of pathological conformation, by means of the method as defined above.

The cell clone of the invention allows the production, which is stable over time, of a synthetic and standardised infectious material of prion type, which is immediately available since it was detected in the cytosol (lysate) and in the supernatant of an infected culture.

### FIGURE LEGEND

The FIGURE is an autoradiogram showing the detection of the PrPsc in the cell lysate of 13 clones (numbered from 1 to 13) with MW: molecular weight indicator.

### DETAILED DESCRIPTION OF THE INVENTION

Definitions

The term “MovS6 line” denotes a cell line expressing the PrP prion protein, originating from an isolation of cells carried out from a dorsal root ganglion of a transgenic mouse overexpressing the ovine PrP gene. The transgenic mouse overexpressing the ovine PrP gene, from which the MovS6 cell line originates, is derived from a cross between a tg301 mouse overexpressing the ovine PrP gene (Vilotte et al. (2001), J. Virol. Vol. 75, p 5977-5984) and a prp0/0 mouse expressing the SV40 T antigen (Tag) (Schwarz et al. (1991), Bio Cell vol. 73: 7-14). The production of the MovS6 cell line is described in the document Archer et al. (2004), J. Virol., p. 482-490.

The MovS6 cell line is a heterogeneous population of cells, not only in terms of functional properties, but also of biological properties. In fact, the dorsal root ganglion from which it was isolated combines different cell populations, in particular glial cells, various types of neurons, etc.

In the context of the invention, the expression “cell clone derived from the MovS6 line” represents every daughter cells derived by cell divisions from a single parent cell belonging to the MovS6 line, and having a genetic inheritance identical to that of the parent cell.

The clones can be isolated by means of a technique known to those skilled in the art, for example by limiting dilution or by flow cytometry, this list not being limiting.

A “PrP prion protein” is generally a sialoglycoprotein anchored at the plasma membrane by a phosphatidyl glycolipid (GPI), naturally present in the cells and involved in the normal function thereof. The normal form of the protein, i.e. the nonpathological form, is generally called Prpc. It is involved in nervous system development in the embryo. In adults, it is expressed essentially in the brain and the spinal cord (neurons and glia). It is involved in cell differentiation and adhesion processes. It also appears to have an antioxidant protective role and a protective role with respect to programmed cell death (apoptosis). This protein also appears to have a role in the folding of other proteins.

The “pathological form PrPsc of the PrP” generally denotes an isoform of the nonpathological protein and represents the marker for prion diseases. This pathological form, associated with prion protein physicochemical properties, notably gives rise to greater resistance to customary disinfecting and sterilising means (heat, chemical products, enzymes, etc). The pathological prion protein thus acquires self-aggregating capacities and can thus form deposits, in particular in the brain, which cause neuron death. The agent responsible for the replication or propagation of the pathological prion protein appears to be the pathological prion protein itself since it is capable of “propagating or multiplying exponentially”, deforming the healthy prion proteins into pathological prion proteins. The “pathological” form of the PrP is therefore the form of the protein of which the conformation is correlated with the appearance of a TSE in infected human or non-human animals.

The expression “capable of withstanding the replication or propagation of the pathological form PrPsc of said PrP” denotes the ability of the cell clone of the invention to produce pathological prion proteins from nonpathological prion proteins, converted into pathological proteins in the presence of the NCTA. The pathological form of the prion protein produced is detected in the cytosol (lysate) or in the culture supernatant. All or part of this cytosolic pathological form can be recovered in the cell lysate.

The “NCTA infection marker titre” is determined by the dose of infectiousness in a sample which allows infection in 50% of inoculation assays. The dose of infectiousness is visualised by the titre of an infection marker, for example PrP.

The infection marker titre can be determined by the titration method described in document WO 04/02179.

The infection marker titre can also be determined by methods well known to those skilled in the art. For example, serial dilutions of the material to be titrated are injected intracerebrally, in a proportion of one dilution of the material per group of animals. According to the incubation time of the disease in each group of animals and the number of animals affected, it is possible to deduce therefrom an infectious titre by means of statistical methods known to those skilled in the art, for instance and preferably the Karber method or else the Spearman-Karber method.

The expression “stability of the infection marker titre” denotes a productivity in terms of infectious agent (PrPsc) which does not significantly decline over time, i.e. no significant loss of productivity can be measured. A significant loss is a decrease in infectious agent productivity of greater than or equal to 1 log₁₀ uWB/ml.

A “passage” is generally the subculturing of all or part of the cells of a culture dish in another dish containing new culture medium. Each passage makes it possible to check the numbers of a cell population and to provide said population with a suitable amount of substrate.

In one particular embodiment, the cell clone of the invention is characterized in that its titre with respect to marker for infection with a non-conventional transmissible agent (NCTA) is stable at least up to the 6^{th} passage, preferably over a period at least between the 6^{th} and 100^{th} passage. Preferably, the titre of the cell clone of the invention with respect to marker for infection with an NCTA is stable at least up to the 7^{th} passage, more preferably at least up to the 8^{th} passage, more preferably at least up to the 9^{th} passage, more preferably at least up to the 10^{th} passage, more preferably at least up to the 11^{th} passage, more preferably at least up to the 12^{th} passage, more preferably at least up to the 13^{th} passage, more preferably at least up to the 14^{th} passage, more preferably at least up to the 22^{nd} passage, more preferably at least up to the 23^{rd} passage, more preferably at least up to the 45^{th} passage, more preferably at least up to the 46^{th} passage, more preferably at least up to the 47^{th} passage, more preferably at least up to the 48^{th} passage, more preferably at least up to the 49^{th} passage, and more preferably at least up to the 50^{th} passage.

The term “TCID50” denotes the infectious dose which causes the infection of 50% of the inoculations tested. These doses are preferably measured by means of the TCIA process.

The term “uWB” (Western blotting unit) denotes an amount of PrPsc detectable by Western blotting.

Generally, there is a difference in sensitivity between the Western blotting and TCIA titration methods. Given this difference, an infection marker titre greater than or equal to 2 log₁₀ Western blotting units/million cells (uWB/million cells), preferably greater than or equal to 3 log₁₀ uWB/million cells, is equivalent to an infectious titre of greater than or equal to 3.5 log 10, preferably greater than or equal to 5 log₁₀ TCID₅₀/ml.

In the context of the invention, the term “NCTA” represents any non-conventional transmissible agent, such as those responsible in human beings for familial or sporadic CJD, for Kuru disease or for variant CJD, or else those responsible in animals for natural

TSEs, such as ovine scrapie, bovine or feline spongiform encephalopathy, chronic wasting disease in cervids or spongiform encephalopathy in mink, or, finally, TSE strains experimentally adapted to laboratory animals. In the context of the invention, the NCTA is also denoted by the term “infectious agent”.

The term “sample” denotes any source of material which may be contaminated with an NCTA. Such a source of material may, for example, be a liquid, a food product, a drink, a cosmetic product or a product derived from genetic engineering, a molecule capable of inhibiting the infectiousness of an NCTA, this list not being limiting. Preferably, it is a biological sample, for example a biological fluid or a tissue or tissue extract. Such a tissue may be a brain tissue, a vertebral column tissue or a tonsil tissue, this list not being limiting. The sample may also be a composition derived from a human or animal source, such as growth hormone or cell extracts, for instance pituitary extracts. Such a composition can in fact be contaminated with an NCTA. In the case of a biological fluid, the latter may be blood, lymph, urine or milk, this list not being limiting. Preferably, the sample is a blood product or a derivative, for example a plasma derivative or a plasma protein concentrate.

The cell clone of the invention can advantageously be used in an in vitro method for detecting and/or titrating the infectiousness of a non-conventional transmissible agent (NCTA), the marker of which is a protein of pathological conformation, in a sample.

Bringing the Cell Clone Into Contact with the Test Sample:

The cell clone of the invention is brought into contact with the test sample that may be infected with an NCTA, or with an infectious material containing the NCTA as reference material, for example extracts of brains from animals infected with an NCTA, such as an ovine prion. The bringing into contact is carried out according to methods known to those skilled in the art (see WO/2005/022148 or also, for example, Archer et al. Journal of Virology, January 2004, p. 482-490).

The cell clone is then cultured for one or more passages in order to allow the NCTA to replicate or to propagate.

Advantageously, the cell clone can be brought into contact with at least one dilution of the sample that may be infected with the NCTA, in a biologically acceptable aqueous solution, in particular with several dilutions, most particularly with serial or successive dilutions.

These dilutions make it possible to refine the quantification of the infectiousness in the sample tested.

Several clone replicates can be brought into contact with the same dilution of the biological product, in order to give the results a finer statistical resolution.

Culturing the Cell Clones (Step ii)):

The step of culturing the cell clones potentially infected with the biological product, according to any technique known to those skilled in the art, is required for the replication of the NCTA, and therefore for amplifying an amount of NCTA which is initially insufficient to be detected.

In one exemplary embodiment, step ii) of culturing the cell clones, performed so as to amplify the amount of NCTA present in said biological sample by replication of the NCTA, is carried out in DMEM (Dulbecco's Modified Eagle's Medium)+Ham-F12 medium (Life Technologies, Cergy Pontoise, France) (4:1) supplemented with glutamine and foetal calf serum (5% final concentration). The cells are incubated at 37° C. under 5% CO₂. One passage of the cells (with a split ratio of 1 to 10, i.e. 1 cell out of 10 put back in culture) is carried out each week.

The product resulting from the culturing of the cell clones contains the PrP in its pathological form, the amount of which is greater than the amount initially present in the biological sample, if said sample contains the same. The PrP in its pathological form and the NCTA accumulate in the infected cells, and are then excreted into the culture medium or exposed at the surface of the cells.

Culturing step ii) can be carried out for one or more passages, for example between 2 and 10 passages, preferably between 4 and 10 passages.

Determination of the Presence and/or of the Amount of Prion or of NCTA (Step iii)):

The process can directly comprise a step of determining the presence and/or the amount of prion or of NCTA (step iii)), or be coupled to bringing into contact with a source of substrate for the PrP and/or the NCTA.

Bringing Into Contact with a Source of Substrate for the PrP and/or the NCTA (Step a)):

Step ii) of the method of the invention can be followed by bringing the product resulting from the culturing of the cells into contact with a source of substrate for the nonpathological conformer of the PrP and/or the NCTA amplified during step ii).

This source of substrate can be provided, for example, in the form of a product of animal origin, for example a healthy brain homogenate, or else of material derived from in vitro culture. This material can, for example, be derived from cells, such as MovS or N2A (Weissmann et al. (2003), PNAS vol.100 no. 20 p. 1666-11671), Roy for example, or else yeasts, mycetes or bacteria, this list not being limiting. This material derived from in vitro culture can be expressed in various cell compartments, such as the extracellular compartment (for example the supernatant, exosomes, this list not being limiting) and/or membrane and/or cytosolic compartments (cell lystate, for example). The function of this step is to allow the in vitro amplification of the PrPsc and/or of the NCTA harvested during step ii) accompanied by the conversion of the nonpathological form of PrP (contained in the substrate) into the pathological form, autoconversion of the nonpathological form being theoretically impossible. The PrP in its pathological form thus initiates the transformation of the nonpathological form into the pathological form.

At the end of the conversion reaction, the unconverted nonpathological form is not detected by the detection system, as will be explained below.

Incubation of the Reaction Medium and/or Amplification of the NCTA (Step b))

In one preferred embodiment, step (a) is followed by an incubation of the product of the cell culture of step (ii) with a source of substrate for the PrP and/or the NCTA for a period of time sufficient to allow at least a part of the proteins which have a nonpathological form to be transformed into a pathological form of the protein, and to allow the NCTA to amplify. Preferably, each incubation step is carried out for a period of time of between 10 seconds and 4 hours, preferably between 20 minutes and 1 hour, and particularly preferably of 30 minutes.

The amplification medium is advantageously constituted of a buffer (1× PBS, 150 mM NaCl, 1% Triton) supplemented with substrate for nonpathological PrP (for example having a volume 10 times greater than the volume of the sample to be amplified).

Aggregate Separation:

It is found that the proteins converted into pathological forms (PrP^{sc} type) can aggregate with one another and with other particles of pathological form (PrP^{sc}), preventing the conversion of other proteins of nonpathological form into the pathological form. This is a drawback since the method could thus be slowed down by the low number of “conversion foci” present in the reaction medium.

Thus, step c) of the method of the invention consists of the disaggregation of the aggregates possibly formed during the preceding steps, so as to release the particles of PrP of pathological form so that they can convert other nonpathological proteins.

Many methods can be used to disaggregate the aggregates during step c) of the method of the invention. By way of example, mention may be made of treatment with a solvent (such as sodium dodecyl sulphate, dimethyl sulphoxide, acetonitrile, guanidine, urea, trifluoroethanol, dilute trifluoroacetic acid, dilute formic acid, this list not being limiting), modification of the physicochemical characteristics of the solution, such as the pH, the temperature, the ionic strength or the dielectric constant, and also physical methods, such as sonication, laser irradiation, freezing/thawing, autoclave incubation, high pressure, mild homogenisation, or alternatively other sources of irradiation, this list not being limiting. Sonication is preferably used. Sonication is a method known to those skilled in the art and often used in methods for purifying PrP^{sc}, by making it possible to increase aggregate solubility.

The disaggregation is carried out for a period of time sufficient to allow at least a part of the aggregates formed during the bringing into contact in step iii) and the incubation/amplification. It is possible that not all the aggregates will be disaggregated during the implementation of a single disaggregation step. In this case, the concentration of pathological proteins increases over the course of the disaggregation steps.

The duration of the disaggregation step can be readily determined by those skilled in the art, and it can depend on the disaggregation method selected. Preferably, the duration of the disaggregation step is between 1 second and 60 minutes, more preferably between 5 seconds and 30 minutes, and more particularly between 5 seconds and 30 seconds.

Advantageously, steps a) to c) are repeated at least twice, preferably between 5 and 100 times, preferably between 20 and 60 times.

This cycle of steps iii) to v) repeated between 2 and 100 times constitutes a series of amplification cycles. A series can also be repeated several times. In this case, the new series will be initiated starting from a volume of the preceding series in place of the initial NCTA sample.

Protein Detection:

Step d) of detecting the pathological PrP proteins can be carried out by any method known to those skilled in the art. The specific detection of the PrPsc can be carried out by means of a first step of separating the two isoforms PrPc and PrPsc. This separation is carried out on the basis of biochemical properties of the PrPsc which make it possible to distinguish it from the nonpathological proteins, in particular the fact that the PrPsc is resistant to protease-based treatments and is less soluble, or insoluble, even in the presence of detergents. Thus, the first step after the amplification is preferably the separation of the PrPc (nonpathological soluble normal form of PrP) from the sample, which can be carried out, for example, by means of protease treatment, for example proteinase K treatment, or by centrifugation in order to separate the soluble forms (PrPc) from the insoluble forms (PrPsc).

In one particular embodiment, digestion with proteinase K (PK) is a step prior to Western blotting, since it results in the digestion mainly of the PrPc and of little or no PrPsc. It is in fact a property of the PrPsc that it is more resistant to PK compared with PrPc. The subsequent detection step therefore no longer detects the nonpathological form of the protein since said form has been digested by the protease.

The detection step can be carried out using the following methods: immunocytochemistry (for example by cell labelling or Facscan) or immunochemistry (such as Western blotting or an ELISA assay), a process of immunoblotting after an SDS-PAGE step, a radioactivity assay, a fluorescence assay, electron microscopy, and a turbidimetry test for detecting aggregates, and also structural tests including NMR (nuclear magnetic resonance), circular dichroism, Raman spectroscopy, UV absorption, a monoclonal antibody which recognises the pathological form of the protein, this list not being limiting.

It is also possible to detect the pathological form of the proteins by means of an antibody which specifically recognises the pathological form and not the nonpathological form. This antibody can itself be labelled in order to make it easier to detect. Such an antibody may, for example, be the 15B3 antibody (Korth et al., 1997, Nature 1997 Nov. 6, 390 (6655): 74-7). The use of such an antibody is relevant for detecting by flow cytometry the prion protein present on the surface of living infected cells.

NCTA Titration:

The detection of the pathological form of the PrP can be combined with a determination of the amount of PrPsc present in the sample.

The titration can be carried out by means of any titration method known to those skilled in the art. In particular, it can be carried out according to the model of the methods described in documents WO2005022148 and WO2006117483 (in particular the A and B reference examples).

All the results of Western blotting for the various dilutions and the various replicates can be analysed by means of a statistical method known to those skilled in the art which makes it possible to establish an infectious titre, for instance the Spearman-Karber method (Schmidt N. J., Emmous R. W., Diagnostic Procedures for viral, ricketsial and chlaveydial Infection, 1989, 6^{th} edition).

In one embodiment of the invention, the method for calculating the titre is the Spearman-Karber method. This method assumes dilution of the test sample according to a geometric progression, i.e. in a constant proportion between the successive dilutions, and inoculation of a constant volume (in general 0.150 ml) of each dilution in at least five wells. The dilution factor most commonly used is the decimal factor.

In order for the Spearman-Karber formula to be applicable, it is necessary to use a constant number of wells inoculated with each dilution, a constant dilution factor and a range of dilutions which is sufficiently broad to encompass both the dilutions on either side of which one hundred percent of the wells will give a positive reaction, and the dilutions on either side of which one hundred percent of the wells will give a negative reaction.

If one or more of these conditions is (are) not met, it is sometimes assumed that, for a constant dilution factor, the higher or lower dilution coming after the final dilution carried out would have given the desired result. Such a “fabrication” of data is not based on any theoretical foundation, but, if it is applied with sufficient care, it is not dangerous. However, it is preferable to repeat the titration with a more suitable range of dilutions, which is essential if there are serious gaps in the data.

According to the Spearman-Karber formula:

Log₁₀ median dose=(X₀)−(d/2)+dS(rᵢ/nᵢ)

where:

X₀=log₁₀ of the reciprocal value of the lowest dilution at which all the test inocula are positive.

d=log 10 of the dilution factor, also referred to as “dilution step” (i.e. the difference between the dilution logarithm intervals).

nᵢ=number of test inocula used at each dilution.

rᵢ=number of positive test inocula (out of ni).

S (rᵢ/nᵢ)=S (P)=sum of the proportion of positive tests beginning with the lowest dilution giving one hundred percent of positive results.

The summation begins at the X₀ dilution.

The estimated standard deviation is calculated using the following formula:

Log standard deviation=d*√(E(p*(1−p)/(ni−1)))

with p=rᵢ/nᵢ=proportion of positive tests (i.e. of wells of inoculum exhibiting a reaction) at each dilution.

The method of the invention makes it possible to quantify the NCTA-related infectiousness over a range of 4 log, i.e. of 10,000 in vitro infectious units. This can, for example, make it possible to meet the criteria for validation of the effectiveness of processes for obtaining biological products, with respect to the elimination of NCTAs.

Applications of the Method for Detecting or Titrating NCTA-Related Infectiousness:

The invention also relates to the application of the titration method according to the invention in an in vitro method for evaluating and/or checking a process for obtaining or treating a biological product that may be contaminated with an NCTA. This evaluating and/or checking method is characterized in that a titration method according to the invention, as described above, is applied to the biological product, upstream and downstream of said process, and in that the two titre values obtained are compared. By comparing between the two measurements, the degree of elimination of the NCTA or the NCTA reduction factor is determined.

In particular, the titration method according to the invention has the ability to be readily applicable in any type of process for obtaining or purifying biological products, in particular blood products, such as blood plasma derivatives, using for example chromatographies or nanofiltration, in particular the chromatographies described in documents EP 0 3 59 593 and WO 02/092632.

Thus, the implementation of the method of the invention makes it possible to evaluate and/or check the effectiveness of a process (or of a part of a process) for obtaining or treating, or even purifying, any biological product that may be contaminated with an NCTA, in terms of the elimination of this NCTA, by virtue of a titration using specific transgenic cell lines that promote replication of the NCTA, and that are brought into contact with an infectious or potentially infectious material containing the test NCTA. The amounts of NCTA are measured upstream and downstream of the process (or of the part of the process) of which it is intended to assess the effectiveness with regard to the NCTA. By comparing the two measurements, the degree of elimination of the pathogenic agent is determined. Thus, the implementation of the present method can be carried out during a process for obtaining a biological product or in the context of a treatment for eliminating the NCTA following the obtaining of the biological product.

The invention also relates to the application of the titration method according to the invention in an in vitro method for evaluating and/or checking a procedure for decontamination of a material. In this case, the NCTA titre of a biological product containing an NCTA is determined by means of the titration method according to the invention. This infected biological product is then brought into contact with the decontamination material and then the decontamination procedure is applied to this material. Finally, the titre of the biological product having undergone the decontamination procedure is again determined. The two titre measurements carried out upstream and downstream of the decontamination procedure are compared so as to evaluate the effectiveness of the decontamination procedure. The material may, for example, be a purification material, in particular a chromatography column, or else be the sanitising of a chromatography column using sodium hydroxide.

The invention also relates to the application of the titration method according to the invention in a procedure for selecting and/or a method for evaluating a compound which makes it possible to reduce the titre of the infectious material. In this case, the NCTA titre of a biological product containing an NCTA is determined by means of the titration method according to the invention. This infected biological product is then brought into contact with the test compound and then the titre of the biological product having undergone the decontamination procedure is again determined. The two titre measurements carried out upstream and downstream of the bringing into contact of the sample with the test compound are compared.

The invention also relates to the application of the titration method according to the invention in a procedure for selecting and/or a method for evaluating a compound capable of modulating the infectiousness of an NCTA. In this case, the NCTA titre of a biological product containing an NCTA is determined in the presence and then in the absence of the compound to be evaluated, by means of the titration method according to the invention. The modes of bringing into contact with the compound are determined according to whether the action of the compound prevents the initiation of an infectious cycle or blocks an already initiated infectious cycle. In any event, the titre of the biological product is determined with and without treatment with the test product. The two titre measurements carried out are compared in order to evaluate the modulatory activity of the compound on the infectiousness of an NCTA.

The invention also relates to the application of the titration method according to the invention in a method for identifying a compound which makes it possible to modulate the transformation of the nonpathological form into the pathological form of the NCTA, for example the transformation of PrP into PrP^{sc}. In this case, the NCTA titre of a biological product containing an NCTA is determined by means of the titration method according to the invention. This infected biological product is then brought into contact with the test compound and then the titration method of the invention is applied in order to again determine the titre of the biological product. The two titre measurements carried out upstream and downstream of the bringing into contact with the compound are compared in order to evaluate the effectiveness of the test compound.

Infectious and Standardised Material of Prion Type Produced by the Cell Clone

The cell lysate or culture supernatant produced by the cell clone of the invention can be standardised in infectious units per dose by means of methods known to those skilled in the art. Infected cell lysate stocks can, for example, be prepared so as to contain 3 log₁₀ TCID₅₀ per millilitre. The availability of standardised stocks then allows strict comparison of distinct assay results.

In addition, it can be stabilised by freezing, drying, lyophilisation or spray-drying, optionally in the presence of substances known to those skilled in the art and intended for avoiding a loss of infectious titre, according to the mode of stabilisation used.

Advantageously, this cell lysate or culture supernatant can be used as an infecting inoculum for a method for evaluating and/or checking a process for obtaining or treating a biological product that may be contaminated with an NCTA, in particular a method for purifying blood plasma derivatives, more particularly chromatographies or nanofiltration.

Advantageously, this cell lysate or culture supernatant can be used as an infecting inoculum for a method for evaluating and/or checking a procedure for decontamination of a material that may be contaminated with an NCTA.

Advantageously, this cell lysate or culture supernatant can be used as an infecting inoculum for a method for evaluating a compound which inhibits the infectiousness of an NCTA.

Advantageously, the infectious material (i.e. the inoculum prepared from the cell clone of the invention) makes it possible to do away with the use of collections of ovine or bovine brains infected with sheep scrapie or bovine spongiform encephalopathy.

The infectious material can be used as an infecting inoculum in an in vitro method for evaluating and/or checking a procedure for decontamination of a material.

The material may, for example, be a purification material, in particular a chromatography column.

The decontamination procedure may, for example, be the sanitising of a chromatography column using sodium hydroxide.

The invention also relates to the use of the infectious material as an infecting inoculum for a method for evaluating a compound which modulates the infectiousness of an NCTA. In this case, the modulatory capacity with respect to the infectiousness of an NCTA is according to the invention tested in the presence or in the absence of the compound capable of modulating this infectiousness of an NCTA.

The invention also relates to the use of the infectious material as an infecting inoculum for a method for evaluating and/or checking a procedure for containment of infectious material, in particular a procedure and equipment of P3 type.

The method of the invention will be understood more clearly with the aid of the additional description which follows, which does not limit the scope of the invention.

### EXAMPLES

Materials and Methods

1° Cells and Infectious Material:

MovS6 cells (Archer F. et al. 2004) were selected as cells which tolerate the replication of NCTAs, and the 127-S scrapie strain adapted to Tg301 transgenic mice was used as natural infectiousness source (Vilotte J L et al. 2001). The cells were cultured in DMEM/Ham F-12 (3:1) medium supplemented with glutamine (2 mM final concentration) and foetal calf serum (5% final concentration).

The initial infectiousness source consisted of a homogenate of brains from infected mice at 200 mg/ml (batch: LN-3326 at the titre of 6.17 log₁₀ uWB/ml).

2° Culture:

The MovS6 cells were cultured in order to constitute, after 15 days of incubation, 2 primary banks (coded: LC-19 and LC-21). An aliquot of LC-19 was thawed and cultured for 3 passages in order to constitute 3 secondary banks, coded: LC-46, LC-47 and LC-48. An aliquot of LC-46 was thawed and cultured for 2 passages in order to constitute a tertiary bank coded LC-59.

3° Comparison of In Vitro Titration of a Reference Sample with 2 Batches of MovS6 Cells:

An aliquot of the batch LC-46 and LC-59 cells was thawed and maintained in parallel for 2 passages. With each of these batches of cells, titration plates in the 24-well format (2 cm²/well) were then seeded in parallel in a proportion of 100,000 cells per well. After 24 hours of incubation, serial dilutions of the LN-3326 mouse brain homogenate were prepared with culture medium and according to a serial 10-fold dilution step (from 10⁻¹ to 10⁻⁸). For each dilution of the inoculum, 5 wells of each of the batches of cells were infected.

The cells were brought into contact with 150 μl of the various dilutions of the inoculum for 24 hours, and then 1 ml of new culture medium was added. The cells were maintained in culture for a further 72 hours, until the first passage, at which point all the cells were re-seeded into wells of 10 cm² (6-well plate format).

During the continuation of the cell cultures, the culture medium was changed once a week and the cells were subcultured with a 1-to-10 ratio. The cells not re-seeded were stored at −80° C. in the form of a dry pellet. These cell pellets stored at each passage made it possible to investigate the PrPsc produced by the cells. The detection of the PrPsc produced was carried out directly by Western blotting.

4° Detection of the PrPsc in the Cells:

The cell pellet samples were thawed and taken up in 60 μl of PBS. The cells were lysed by sonication for 15″ using a sonication bath (power: 15 W). 20 μl of sonicated cell lysate were removed so as to be treated with proteinase K. The digestion product was denatured and then analysed by polyacrylamide gel electrophoresis under denaturing conditions (SDS-PAGE). The proteins having migrated in the gel were transferred onto a PVDF membrane by electroblotting. The PrPsc present on the membranes was detected by incubation with the 6H4 antibody (Prionics) and then a secondary antibody labelled with alkaline phosphatase (goat “anti-mouse antibody” antibody). The labelled membranes were revealed by chemiluminescence. A sample was considered to be positive if the electrophoretic profile with three glycosylated PrPsc forms was visible on the autoradiograms. Taking into account the dilutions of the sample in the various reagents necessary for the Western blotting, the sample volume actually loaded in the lane of the gel was 4.35 μl.

For each analysis by Western blotting, negative controls (noninfected MovS6 cells) were treated in parallel to the samples. At each of the cell passages, all the culture plate wells were tested. When all the replicates of cell cultures inoculated with a given dilution of the inoculum were found to be positive for the PrPsc during two successive passages, these cultures were no longer tested during the subsequent passages.

5° Reproducibility of an In Vitro Titration:

In order to evaluate the reproducibility of the in vitro titration system, the titration of the LN-3326 reference sample was reproduced 2 other times with a new aliquot of the LC-46 cell batch for each titration. The titrations were carried out as described above.

6° Stability of the Production of PrPsc by the Infected MovS6 Cell (Batch LC-46):

In order to evaluate the stability of the production of PrPsc by infected MovS6 cells, 5×10⁵ cells (batch LC-46), having undergone 4 passages after they had been thawed, were seeded in 10 ml of culture medium in a 25 cm² flask and inoculated with a PrPsc load of 2.2 log₁₀ uWB, i.e. with a multiplicity of infection (MOI) of 1:3100. The infected culture was maintained for 23 passages with one passage carried out each week according to a split ratio of 1:10. At each passage, an aliquot of 3×10⁶ cells, from the cells that were not re-seeded, was stored at −80° C. in the form of a dry pellet so as to make it possible to carry out, at the end of the assay, a titration by Western blotting of the PrPsc in the cell lysates harvested at various passages.

The PrPsc was assayed by limiting dilution of the aliquot and analysis by Western blotting as described in section 4°. The first dilution of the sample no longer exhibiting any signals specific for the PrPsc was considered to contain 1 Western blotting unit. The titre of the sample was then calculated as the inverse of the limiting dilution, taking into account the volume of the aliquot actually loaded on the electrophoresis gel. Thus, 3×10⁶ cells taken up in 60 μl of PBS corresponded to 3×10⁶ cells/0.06 ml, i.e. 50×10⁶ cells/ml. Under the Western blotting (WB) analysis conditions described in section 4, the signal observed on the lane corresponding to the “nondiluted” sample in the dilution range originated from 4.35 μl of suspension of cell pellet taken up in PBS and not diluted, i.e. 0.22×10⁶ cells. By way of example, if the first dilution at which the signal was no longer detected corresponds to the 1/81^{st} dilution, the PrPsc titre in the cell pellet was 81 uWB/4.35 μl, i.e. 81×1000/4.35=18620 uWB/ml, i.e., expressed as decimal logarithm, 4.27 log₁₀ uWB/ml, or alternatively 81/0.22 uWB/million cells, i.e. 368 uWB/million cells, i.e., expressed as decimal logarithm, 2.57 log₁₀ uWB/million cells.

Results

The results of the titrations of the reference brain homogenate (batch LN-3326) with each of the batches of MovS6 cells (LC-46 and LC-59) are summarised in Tables 1 and 2.

TABLE NO. 1
Titration of the reference infected mouse brain
homogenate (LN-3326) with the LC-59 cell batch

| | Number of infected cultures according to the dilution | | | | | | Titre |
|---|---|---|---|---|---|---|---|
| Pas- | inoculated (infected wells/inoculated wells) | | | | | | (log |
| sage | | | | | | Neg. | TCID₅₀/ |
| No. | 10⁻¹ | 10⁻² | 10⁻³ | 10⁻⁴ | 10⁻⁵ | control | ml) |
| 1 | 0/5 | 0/5 | 0/5 | 0/5 | 0/5 | 0/5 | <0.3 |
| 2 | NT | 0/5 | 0/5 | 0/5 | 0/5 | 0/5 | ≧2.3 |
| 3 | 5/5 | 0/5 | 0/5 | 0/5 | 0/5 | 0/5 | 2.3 |
| 4 | 5/5 | 0/5 | 0/5 | 0/5 | 0/5 | 0/5 | 2.3 |
| 5 | NT | 5/5 | 0/5 | 0/5 | 0/5 | 0/5 | 3.3 |
| 6 | NT | 5/5 | 0/5 | 0/5 | 0/5 | 0/5 | 3.3 |
| 7 | NT | 5/5 | 0/5 | 0/5 | 0/5 | 0/5 | 3.3 |

NT: Not tested

TABLE NO. 2
Titration of the reference infected mouse brain
homogenate (LN-3326) with the LC-46 cell batch.

| | Number of infected cultures according to the dilution | | | | | | Titre |
|---|---|---|---|---|---|---|---|
| Pas- | inoculated (infected wells/inoculated wells) | | | | | | (log |
| sage | | | | | | Neg. | TCID₅₀/ |
| No. | 10⁻² | 10⁻³ | 10⁻⁴ | 10⁻⁵ | 10⁻⁶ | control | ml) |
| 1 | 0/5 | 0/5 | 0/5 | 0/5 | 0/5 | 0/5 | 0 |
| 2 | 5/5 | 0/5 | 0/5 | 0/5 | 0/5 | 0/5 | 3.3 |
| 3 | 5/5 | 3/5 | 0/5 | 0/5 | 0/5 | 0/5 | 3.9 |
| 4 | 5/5 | 5/5 | 4/5 | 0/5 | 0/5 | 0/5 | 5.1 |
| 5 | NT | 5/5 | 5/5 | 5/5 | 0/5 | 0/5 | 6.3 |
| 6 | NT | NT | 5/5 | 5/5 | 0/5 | 0/5 | 6.3 |
| 7 | NT | NT | 5/5 | 5/5 | 0/5 | 0/5 | 6.3 |

NT: Not tested

The results of the repeated titration of the LN-3326 reference sample are summarised in Tables 3 and 4 below:

TABLE NO. 3
Repetition of the titration of the reference infected mouse
brain homogenate (LN-3326) with the LC-46 cell batch

| | Number of infected cultures according to the dilution | | | | | | | Titre |
|---|---|---|---|---|---|---|---|---|
| Pas- | inoculated (infected wells/inoculated wells) | | | | | | | (log₁₀ |
| sage | | | | | | | Neg. | TCID₅₀/ |
| No. | 10⁻² | 10⁻³ | 10⁻⁴ | 10⁻⁵ | 10⁻⁶ | 10⁻⁷ | control | ml) |
| 1 | 2/5 | 0/5 | 0/5 | 0/5 | 0/5 | 0/5 | 0/5 | 2.7 |
| 2 | 5/5 | 0/5 | 0/5 | 0/5 | 0/5 | 0/5 | 0/5 | 3.3 |
| 3 | 5/5 | 5/5 | 0/5 | 0/5 | 0/5 | 0/5 | 0/5 | 4.3 |
| 4 | 5/5 | 5/5 | 5/5 | 0/5 | 0/5 | 0/5 | 0/5 | 5.3 |
| 5 | NT | 5/5 | 5/5 | 0/5 | 0/5 | 0/5 | 0/5 | 5.3 |
| 6 | NT | 5/5 | 5/5 | 3/5 | 1/5 | 0/5 | 0/5 | 6.1 |
| 7 | NT | 5/5 | 5/5 | 3/5 | 2/5 | 0/5 | 0/5 | 6.3 |
| 8 | NT | 5/5 | 5/5 | 4/5 | 2/5 | 0/5 | 0/5 | 6.5 |
| 9 | NT | NT | 5/5 | 4/5 | 2/5 | 0/5 | 0/5 | 6.5 |

NT: Not tested

TABLE NO. 4
Repetition of the titration of the reference infected mouse
brain homogenate (LN-3326) with the LC-46 cell batch

| | Number of infected cultures according to the dilution | | | | | | | Titre |
|---|---|---|---|---|---|---|---|---|
| Pas- | inoculated (infected wells/inoculated wells) | | | | | | | (log₁₀ |
| sage | | | | | | | Neg. | TCID₅₀/ |
| No. | 10⁻² | 10⁻³ | 10⁻⁴ | 10⁻⁵ | 10⁻⁶ | 10⁻⁷ | control | ml) |
| 3 | 5/5 | 5/5 | 0/5 | 0/5 | 0/5 | 0/5 | 0/5 | 4.3 |
| 4 | 5/5 | 5/5 | 3/5 | 0/5 | 0/5 | 0/5 | 0/5 | 4.9 |
| 5 | NT | 5/5 | 5/5 | 0/5 | 0/5 | 0/5 | 0/5 | 5.3 |
| 6 | NT | 5/5 | 5/5 | 3/5 | 1/5 | 0/5 | 0/5 | 6.1 |
| 7 | NT | 5/5 | 5/5 | 4/5 | 1/5 | 0/5 | 0/5 | 6.3 |
| 8 | NT | 5/5 | 5/5 | 4/5 | 1/5 | 0/5 | 0/5 | 6.3 |

NT: Not tested

During each of these titration assays, no trace of infectiousness was observed in any of the 5 replicates of cells inoculated with the noninfected brain homogenate sample (neg. control). Conversely, an increasing PrPsc production was observed with the cells inoculated with the LN-3326 reference sample. These results confirm that the in vitro titration system makes it possible to demonstrate a de novo PrPsc production by infected cells.

The LN-3326 reference homogenate sample during the repetitions of the titration with the LC-46 cell batch shows an average infectious titre of 6.4 log₁₀ TCID₅₀/ml (range 6.3 to 6.5 log₁₀ TCID₅₀/ml).

The LN-3326 reference homogenate sample during the titration with the LC-59 cell batch shows an infectious titre of 3.3 log₁₀ TCID₅₀/ml.

The infectious titre of the reference sample, measured with the 2 cell batches: LC46 and LC59, shows a difference of 3.1 log₁₀. This difference is significant.

This difference in titre indicates that the infectious titre is dependent on the MovS6 cell batch used for the in vitro titration.

7° Stability of PrPsc Production by MovS6 Cells:

The kinetics of the PrPsc titre measured in a constant amount of inoculated MovS6 cells is summarised in Table 5.

TABLE 5
PrPsc titre measured in an infected cell lysate

| Passage number (PI) | 6 | 10 | 12 | 17 | 19 | 20 | 22 | 23 |
|---|---|---|---|---|---|---|---|---|
| Titre in log₁₀ (uWB/ml) | 6.2 | 6.2 | 5.7 | 5.7 | 5.2 | 5.7 | 4.75 | 4.75 |

Legend: PI: post-inoculation

These data indicate an average titre of 5.5 log₁₀ uWB/ml. The standard deviation (σ) is ±0.6 log₁₀ uWB/ml. The variation in PrPsc titre in the cell lysate over time, expressed by the standard deviation, is greater than the variability of the Western blotting titration method, of ±0.5 log₁₀. These data therefore indicate a significant decrease in the titre in the lysate of cells harvested between the 6^{th} passage and the 23^{rd} passage after inoculation.

8° Subcloning:

An aliquot of LC-46 was thawed and cultured for 8 days. From this culture, five 96-well plates were seeded in a proportion of 0.3 cell/well. After 37 days of culture, 13 clones were isolated and re-seeded (split ratio: 100%) onto a 24-well plate (2cm²/well). After 8 days of incubation, each of these cultures was divided up into 2 batches. The first was maintained in a 25 cm² flask until the constitution of a pre-bank (LC-82-1 to LC-82-13) and the other was deposited on a 24-well plate in order to be infected as follows: each clone culture was adjusted to 100,000 cells/well and then inoculated with an identical load of PrPsc of 1.9 log₁₀ uWB. These cultures were maintained for 6 passages. At the end of this period, the cells of each culture were harvested and adjusted to a concentration of 10⁶ cells/ml. These cell suspensions were stored at −80° C. in the form of a dry pellet.

8.1° Clone selection:

In order to evaluate, by Western blotting, the strength of the response to the infection by each of the clones, an aliquot of dry pellet of each of the clones was thawed, sonicated and analysed by Western blotting. The detection of the PrPsc in the various clone lysates is represented on the autoradiogram (FIGURE).

These data show that the response to the infection by each of the clones, measured 6 weeks after the inoculation, is heterogeneous between the clones.

The MovS6-13 and -10 clones show no PrPsc-specific signal.

The MovS6-2-3-5-7-8-9-12 clones show a PrPsc-specific signal of medium intensity.

The MovS6-1-6-11 clones show a PrPsc-specific signal of strong intensity.

The MovS6-4 clone shows a PrPsc-specific signal of very strong intensity.

The corresponding aliquot of noninfected cells (LC-82-4) was thawed and maintained in culture in order to constitute a primary bank (LC-91) and 2 secondary banks (LC-93 and LC-94).

8.2° Stability of PrPsc Production by the Cells of the MovS6-4 Clone:

In order to evaluate the stability of PrPsc production by infected MovS6-4 cells, 5×10⁵ cells, having undergone 2 passages after they had been thawed, were seeded in 10 ml of culture medium in a 25 cm² flask and inoculated with a PrPsc load of 3.0 log₁₀ uWB, i.e. with a multiplicity of infection (MOI) of 1:500. The infected culture was maintained for 37 passages with a passage being carried out each week according to a split ratio of 1:10. At each passage, an aliquot, from the cells not re-seeded, was stored at −80° C. in the form of a dry pellet in order to make it possible to carry out, at the end of the assay, a Western blotting titration of the PrPsc in the cell lysates harvested at various passages. At the end of this assay, the PrPsc in the lysates was titrated by limiting dilution and Western blotting as described in section 6°.

8.3° Comparison of In Vitro Titration of a Reference Sample with the MovS6 and MovS6-4 Cells:

An aliquot of MovS6-4 cells (LC-94) was thawed and maintained for 5 passages. Titration plates in the 24-well format (2 cm²/well) were then seeded in a proportion of 100,000 cells per well. After 24 hours of incubation, serial dilutions of the LN-3326 mouse brain homogenate were prepared with culture medium and according to a serial 10-fold dilution step (from 10⁻¹ to 10⁻⁸). For each dilution of the inoculum, 5 wells of cells were infected. The cells were brought into contact with 150 μl of the various dilutions of the inoculum for 24 hours, and then 1 ml of new culture medium was added. The cells were maintained in culture for a further 72 hours, up to the first passage, at which point all the cells were re-seeded in wells of 10 cm² (6-well plate format).

During the continuation of the cell cultures, the culture medium was changed once a week and the cells were subcultured with a ratio of 1 to 10. The cells not re-seeded were stored at −80° C. in the form of a dry pellet. These cell pellets stored at each passage made it possible to investigate the PrPsc produced by the cells. The detection of the PrPsc produced was carried out directly by Western blotting as described in section 4°.

Results

The kinetics of the PrPsc titre measured in a constant amount of inoculated MovS6-4 cells is summarised in Table 6.

TABLE 6
PrPsc titre (log₁₀) measured in the infected cell lysate

| Passage PI | 6 | 8 | 9 | 10 | 11 | 14 | 17 | 18 |
|---|---|---|---|---|---|---|---|---|
| Titre (uWB/ml) | 4.75 | 4.75 | 5.2 | 5.0 | 4.75 | 4.3 | 4.75 | 4.5 |
| Passage PI | 20 | 23 | 25 | 34 | 36 | 37 | 40 | 47 |
| Titre (uWB/ml) | 4.3 | 4.3 | 4.3 | 3.8 | 4.75 | 4.75 | 4.3 | 4.75 |

These data indicate an average titre of 4.6 log₁₀ uWB/ml. The standard deviation (σ) is ±0.3 log₁₀ uWB/ml. The variation in PrPsc titre in the cell lysate over time, expressed by the standard deviation, is less than the variability of the Western blotting titration method, of ±0.5 log₁₀. These data therefore indicate stability of the titre in the lysate of cells harvested between the 6^{th} passage and the 47^{th} passage after inoculation. Although these data indicate a titre measured by Western blotting and expressed in Western blotting unit (uWB), it can be considered that the infectious titre of the infected cells (cell lysates) is also stable over the period studied. Given the difference in sensitivity between the Western blotting and TCIA titration methods, the infectiousness between the 6^{th} and the 47^{th} passage would be approximately 6.2 log₁₀ TCID₅₀/ml.

The results of the titrations of the reference brain homogenate (batch LN-3326) with the various batches of MovS6 cells and the MovS6-4 clone are summarised in Table 7.

TABLE No. 7
Titration of the reference infected mouse brain homogenate (LN-3326)
with the various batches of MovS6 cells (LC-46, LC-59) and
the MovS6-4 clone (batch LC-94)

| Cell | MovS6 | MovS6 | | | MovS6-4 |
|---|---|---|---|---|---|
| Batch | LC-59 | LC-46 | | | LC-94 |
| TCID₅₀/ml | 3.3 log₁₀ | 6.3 log₁₀ | 6.5 log₁₀ | 6.3 log₁₀ | 4.7 log₁₀ |

The LN-3326 reference homogenate sample during the repetitions of the titration with the MovS6 cells (batch: LC-46) showed an average infectious titre of 6.4 log₁₀ TCID₅₀/ml (range 6.3 to 6.5 log₁₀ TCID₅₀/ml), see Tables 3 and 4.

The LN-3326 reference homogenate sample during the titration with the MovS6-4 cells showed an infectious titre of 4.7 log₁₀ TCID₅₀/ml.

The infectious titre of the reference sample, measured with the 2 cells MovS6 (batch LC-46) and MovS6-4, shows a difference of 1.7 log₁₀. This difference is significant; it confirms that the infectious titre is dependent on the cell used for the in vitro titration.

Conclusions

During this study, 2 batches of cells of the MovS6 line, respectively denoted LC-46 and LC-59, were used. These 2 batches differ in terms of the age of their cells at inoculation. The age of the LC-46 batch is 5 passages and a single freezing/thawing cycle, while the age of the LC-59 batch is 7 passages and 2 freezing/thawing cycles.

The data from titration of the sample of reference brain homogenate (LN-3326) showed that the infectious titre of a sample is dependent on the MovS6 cell batch used. The titration system, TCIA (tissue culture infectivity assay), carried out with the older cells (batch: LC-59) was in fact characterized by a loss of sensitivity compared with the system of titration carried out with the younger cells (batch: LC-46).

Moreover, the data from this study show that, after inoculation of the MovS6 cells (batch LC-46), the PrPsc production declined over time since a significant loss of productivity was observed between the 6^{th} and the 23^{rd} passage post-inoculation.

These two observations in fact show an effect of the age of the cells, expressed as number of passages and of freezing/thawing cycles, on the biological properties of the MovS6 line. This age effect consists of a decrease in the permissivity of the cells to infection and a loss of PrPsc productivity by the infected cells.

The MovS6-1 and MovS6-10 clones show no PrPsc production at the 6^{th} week after inoculation, whereas a high level of production was observed with the MovS6-4 clone.

8.4° Comparison of the Permissivity of the MovS6 and MovS6-4 Cells:

An aliquot of noninfected MovS6-4 cells (LC-82-4) was thawed and maintained in culture in order to constitute a primary bank (LC-91).

Preparation of the Sample Before Infection

An aliquot of MovS6-4 cells (LC-91) was thawed and maintained in culture in order to constitute four secondary banks (LC-131 to LC-134).

An aliquot of MovS6-4 cells (LC-131) was thawed and maintained for 64 days and 10 passages, and then frozen in order to constitute the tertiary bank (LC-139).

An aliquot of MovS6-4 cells (LC-139) was thawed and maintained for 66 days and 10 passages, and then frozen in order to constitute the quaternary bank (LC-143).

Comparison of In Vitro Titration of a Reference Sample with the MovS6 Cells and the MovS6-4 Clone

An aliquot of MovS6-4 cells (LC-143) was thawed and maintained for 2 passages. Titration plates in the 24-well format (2 cm²/well) were then seeded in a proportion of 100,000 cells per well. After 24 hours of incubation, serial dilutions of the LN-3326 mouse brain homogenate were prepared with culture medium and according to a serial 10-fold dilution step (from 10⁻¹ to 10⁻⁸). For each dilution of the inoculum, 5 wells of cells were infected.

The cells were brought into contact with 150 μl of the various dilutions of the inoculum for 24 hours, and then 1 ml of new culture medium was added. The cells were maintained in culture for a further 72 hours, up to the first passage, at which point all the cells were re-seeded into wells of 10cm² (6-well plate format).

During the continuation of the cell cultures, the culture medium was changed once a week and the cells were subcultured with a ratio of 1 to 10. The cells not re-seeded were stored at −80° C. in the form of a dry pellet. These cell pellets stored at each passage made it possible to investigate the PrPsc produced by the cells. The detection of the PrPsc produced was carried out directly by Western blotting as described in section 4° above.

The results of titrations of the reference brain homogenate (batch LN-3326) with the various batches of MovS6 cells and the MovS6-4 clone at the 6^{th} passage after inoculation are summarised in Table 8 below:

TABLE No. 8
Titration of the reference infected mouse brain homogenate (LN-3326)
with the various batches of MovS6 cells (LC-46 and LC-59) and
the MovS6-4 clone (LC-143)

| | Cell line | | | |
|---|---|---|---|---|
| | “young” | “aged” | | |
| | MovS6 | MovS6 | “aged” MovS6-4 | |
| Batch | LC-46 | LC-59 | LC-143 | |
| Number of total | 1 thawing | 2 thawings | 3 thawings and | |
| thawing/ freezing | and | and | 2 freezings | |
| cycles before infection | 1 freezing | 2 freezings | | |
| Number of total | 5 passages | 7 passages | 22 passages | |
| passages before | | | | |
| infection | | | | |
| TCID₅₀/ml | 6.4 log₁₀ | 3.3 log₁₀ | 5.72 log₁₀ | 5.92 log₁₀ |

The “young” MovS6 cells (batch: LC-46) underwent, prior to the titration assay, a single freezing/thawing cycle and a total of 5 passages. With these cells, the LN-3326 reference homogenate sample showed an infectious titre of 6.4 log₁₀ TCID₅₀/ml.

The “aged” MovS6 cells (batch: LC-59) underwent, prior to the titration assay, two freezing/thawing cycles and a total of 7 passages. With these cells, the LN-3326 reference homogenate sample showed an infectious titre of 3.3 log₁₀ TCID₅₀/ml.

The “aged” MovS6-4 cells (batch: LC-143) underwent, prior to the titration assay, two freezing cycles and 3 thawing cycles and a total of 22 passages. With these cells, the LN-3326 reference homogenate sample showed an infectious titre of 5.82 log₁₀ TCID₅₀/ml (range 5.72 to 5.92).

The titre of the reference brain homogenate sample (LN-3326) determined with the “young” MovS6 cells is not significantly different from the infectious titre determined with the “aged” MovS6-4 cells (the difference between the titres is less than the difference threshold of 1 log₁₀ commonly considered to be the threshold valve for a significant difference).

The titre of the reference brain homogenate sample (LN-3326) determined with the “aged” MovS6 cells is significantly different from the infectious titre determined with the “aged” MovS6-4 cells.

Conclusion:

During this study, 2 batches of cells of the MovS6 line, respectively denoted LC-46 and LC-59, were used. These 2 batches differ only in terms of their “age” (expressed by the number of freezing/thawing cycles and the number of passages of the cells before their inoculation).

The age of the LC-46 batch is 5 passages and a single freezing/thawing cycle, while the age of the LC-59 batch is 7 passages and 2 freezing/thawing cycles.

The data from titration of the reference brain homogenate sample (LN-3326) showed that the infectious titre of a sample is dependent on the age of the MovS6 cell used.

The titration system, TCIA (“Tissue Culture Infectivity Assay”), carried out with the aged cells (batch: LC-59), was in fact characterized by a significant loss of sensitivity compared with the system of titration carried out with the younger cells (batch: LC-46).

These observations in fact show the age of the cells, expressed as number of passages and of freezing/thawing cycles, on the biological properties of the MovS6 line. This age effect consists in a decrease in the permissivity of the cells to infection in MovS6.

Moreover, these data also show that, after inoculation of the MovS6-4 cells (batch LC-143), these cells having undergone 2 freezing/thawing cycles and one additional thawing, and a total of 22 passages, the infectious titre of a sample is not affected by the age of this cell. Consequently, the age of the MovS6-4 cells (LC-143) has no impact on the sensitivity of the system of titration by TCIA.

The titration system, TCIA (“Tissue Culture Infectivity Assay”), carried out with the “aged” MovS6-4 cells (batch: LC-143) was in fact characterized by stability of the sensitivity compared with the system of titration carried out with the “young” MovS6 cells (batch: LC-46).

This observation shows that the effect of the age of the cells, expressed as number of passages and of freezing/thawing cycles, has no impact on the permissivity of the cells to infection in MovS6-4.

It can therefore be concluded that the MovS6-4 clone, contrary to the MovS6 cells, is stable in particular with respect to its permissivity to infection by a prion strain.

1. Cell clone derived from the MovS6 line, said cell clone expressing a prion protein PrP and being capable of tolerating the replication or propagation of the pathological form PrPsc of said PrP, wherein its titre with respect to marker for infection with a non-conventional transmissible agent (NCTA) is stable at least up to the 6^{th} passage.

2. The cell clone according to claim 1, wherein its titre with respect to marker for infection with a non-conventional transmissible agent (NCTA) is stable over a period at least between the 6^{th} and the 100^{th} passage.

3. The cell clone according to claim 1, wherein its titre with respect to marker for infection with a non-conventional transmissible agent (NCTA) is stable at least up to the 7^{th} passage.

4. The cell clone according to claim 1, wherein its titre with respect to marker for infection with a non-conventional transmissible agent (NCTA) is stable at least up to the 8^{th} passage.

5. The cell clone according to claim 1, wherein its titre with respect to marker for infection with a non-conventional transmissible agent (NCTA) is stable at least up to the 10^{th} passage.

6. The cell clone according to claim 1, wherein its titre with respect to marker for infection with a non-conventional transmissible agent (NCTA) is stable at least up to the 14^{th} passage.

7. The cell clone according to claim 1, wherein its titre with respect to marker for infection with a non-conventional transmissible agent (NCTA) is stable at least up to the 22^{nd} passage.

8. The cell clone according to claim 1, wherein its titre with respect to marker for infection with a non-conventional transmissible agent (NCTA) is stable at least up to the 23^{rd} passage.

9. The cell clone according to claim 1, wherein its titre with respect to marker for infection with a non-conventional transmissible agent (NCTA) is stable at least up to the 47^{th} passage.

10. The cell clone according to claim 1, wherein the cell lysate or the culture supernatant of said cell clone has, from 6 weeks after infection, an infection marker titre greater than or equal to 3 log₁₀ TCID50 per million cells, preferably greater than or equal to 4 log₁₀ TCID50 per million cells, preferably greater than or equal to 6 log₁₀ TCID50 per million cells, preferably greater than 7 log₁₀ TCID50 per million cells.

11. The cell clone according to claim 1, deposited on 22 Feb. 2008, under deposit number CNCM I-3922, at the Collection Nationale de Cultures des Microorganismes.

12. An in vitro method for detecting and/or titrating the infectiousness of a non-conventional transmissible agent (NCTA), the marker of which is a protein of pathological conformation, in a sample, comprising the steps consisting in:
i) bringing said sample into contact with a cell clone as described in claim 1,
ii) culturing said cell clone in order to amplify the amount of NCTA present in said sample by replication of said NCTA,
iii) determining the presence and/or the amount of pathological protein and/or of NCTA in the sample,
the culturing step ii) being carried out for one or more passages.

13. The method according to claim 12, in which step (iii) comprises the following steps:
a) bringing the NCTA thus amplified at the end of step ii) into contact with a source of substrate for the pathological protein and/or the NCTA,
b) incubating the reaction medium allowing the transformation of the nonpathological conformer into the pathological conformer and/or the amplification of the NCTA,
c) disaggregating the aggregates possibly formed during step i) or ii),
d) determining the presence and/or the amount of pathological protein and/or of NCTA in the sample,
steps (a) to (c) constituting a cycle of operations which is repeated at least twice before step (d).

14. An in vitro method for evaluating and/or checking a process for obtaining or treating a biological product or a material that may be contaminated with an NCTA, in which method a titration method according to claim 12 is applied to said biological product or material, (A) upstream and (B) downstream of said process, and the two titre values (A) and (B) obtained are compared.

15. An in vitro method for evaluating and/or checking a procedure for decontamination of a biological product or of a material, in which method a titration method according to claim 12 is applied to said biological product or material, (A) upstream and (B) downstream of said procedure, and the two titre values (A) and (B) obtained are compared.

16. An in vitro method for evaluating a compound capable of modulating the infectiousness of an infectious biological product, in which method a titration method according to claim 12 is applied to said infectious biological product, (A) in the presence and (B) in the absence of said compound to be evaluated, and the two titre values (A) and (B) obtained are compared.

17. An in vitro method for the diagnosis of a transmissible spongiform encephalopathy in a human individual or a non-human animal individual, which comprises detecting the presence, in a biological sample from said individual, of a non-conventional transmissible agent (NCTA) which is a protein of pathological conformation, by means of the method as defined in claim 12.
